(19) United States
(12) Patent Application Publication
(10) Pub. No.: US 2024/0140981 A1
(43) Pub. Date: May 2, 2024

(54) NUCLEIC ACID PURIFICATION METHODS USING A WATER-IMMISCIBLE SOLVENT WASH

(71) Applicant: Curative Inc., San Dimas, CA (US)

(72) Inventors: Vladimir I. SLEPNEV, Diamond Bar, CA (US); Katie BOND, San Dimas, CA (US); James MISSELBROOK, San Dimas, CA (US); Anna Katherine RODGERS, San Dimas, CA (US)

(21) Appl. No.: 18/269,743

(22) PCT Filed: Dec. 28, 2021
(86) PCT No.: PCT/US2021/065273

§ 371 Date: Jun. 26, 2023

Related U.S. Application Data
(60) Provisional application No. 63/131,734, filed on Dec. 29, 2020.

(51) Int. Cl. C07H 21/00; C07H 1/08

(52) U.S. Cl. CPC C07H 21/00; C07H 1/08

(57) ABSTRACT

The present disclosure, in some aspects, is directed to nucleic acid purification methods using a water-immiscible solvent wash. In other aspects, the present disclosure is directed to kits, components, and compositions (such as consumables) useful for the nucleic acid purification methods described herein.

### CROSS-REFERENCE TO RELATED APPLICATIONS

This application claims priority to and the benefit of U.S. Provisional Patent Application No. 63/131,734, filed on Dec. 29, 2020, which is incorporated herein by reference in its entirety.

### TECHNICAL FIELD

The present disclosure, in some aspects, is directed to nucleic acid purification methods using a water-immiscible solvent wash. In other aspects, the present disclosure is directed to kits, components, and compositions (such as consumables) useful for the nucleic acid purification methods described herein.

### BACKGROUND

Certain components of nucleic acid samples and agents introduced during nucleic acid purification techniques, such as chaotropic salts used to facilitate nucleic acid binding to a capture medium such as silica, are inhibitors of downstream processes including polymerase chain reaction (PCR). Thus, it is critical that a nucleic acid purification method remove inhibitors to the extent possible. As is currently a standard in the field, following binding of a nucleic acid to a solid support, such as a silica-based support, nucleic acid purification methods include several wash steps designed to remove inhibitors prior to nucleic acid elution. For example, as described in Vandeventer et al. and Honsvall & Robertson, nucleic acid purification methods are purposefully designed with numerous lengthy wash steps Phys Chem B, 117, 2013; J Biomol Tech, 28, 2017), resulting in an overall purification process that takes about 45 minutes to 1 hour. Honsvall & Robertson explored reducing the number of wash steps and observed that this resulted in the presence of inhibitors that negatively impacted downstream processes.

### BRIEF SUMMARY

In some aspects, provided is a method of purifying a nucleic acid from a sample, the method comprising: (a) forming a two-phase solution on top of a silica medium, wherein the two-phase solution comprises a first layer comprising an aqueous binding solution and the sample containing the nucleic acid, wherein the two-phase solution comprises a second layer comprising a water-immiscible solvent, and wherein the first layer is in contact with the silica medium and the second layer is on top of the first layer; (b) passing the two-phase solution through the silica medium; (c) loading an elution solution on top of the silica medium; and (d) passing the elution solution through the silica medium to obtain the purified nucleic acid. In some embodiments, forming the two-phase solution comprises loading the first layer on top of the silica medium and then loading the second layer on top of the first layer.

In some embodiments, the water-immiscible solvent comprises a long-chain aliphatic alcohol. In some embodiments, the water-immiscible solvent comprises an octanol, decanol, undecanol, or a mixture thereof. In some embodiments, the water-immiscible solvent comprises dimethyl-octanol.

In some embodiments, the method further comprises obtaining the first layer by mixing the aqueous binding solution and the sample.

In some embodiments, the aqueous binding solution comprises a chaotropic salt and/or a short-chain alcohol. In some embodiments, the chaotropic salt is a guanidinium salt. In some embodiments, the guanidinium salt is guanidinium thiocyanate or guanidinium chloride.

In some embodiments, the short-chain alcohol comprises ethanol, methanol, propanol, isopropanol, or a combination thereof. In some embodiments, the short-chain alcohol comprises ethanol.

In some embodiments, the aqueous binding solution further comprises a reducing agent. In some embodiments, the reducing agent is DTT or TCEP.

In some embodiments, the silica medium is a silica membrane. In some embodiments, the silica medium is a silica-based filter.

In some embodiments, the elution solution is deionized water. In some embodiments, the elution solution is a low ionic strength buffered solution. In some embodiments, the low ionic strength buffered solution is a Tris-base buffer, such as having 100 mM or less Tris.

In some embodiments, the silica medium is positioned in a container, and wherein the container comprises a loading zone above the silica medium and an eluate zone below the silica medium.

In some embodiments, passing the two-phase solution through the silica medium or passing the elution solution through the silica medium comprises subjecting the container to centrifugation or the application of a pressure.

In some embodiments, passing the two-phase solution through the silica medium is completed in a single centrifugation step.

In some embodiments, the method is completed in 20 minutes or less.

In some embodiments, the method does not comprise a drying step between passing the two-phase solution through the silica medium and passing the elution solution through the silica medium.

In other aspects, provided is a composition comprising a nucleic acid obtained from a nucleic acid purification method of any one of the methods described herein.

In some embodiments, the composition described herein comprises at least about 2 fold less of a compound inhibiting nucleic acid amplification as compared to a composition obtained from a nucleic acid purification method not comprising use of a water-immiscible solvent wash.

### BRIEF DESCRIPTION OF THE DRAWINGS

FIG. 1 shows a plot of relative fluorescence units (RFU) per cycle of an amplification reaction of SARS CoV-2 viral RNA.

FIG. 2 shows a plot of relative fluorescence units (RFU) per cycle of an amplification reaction of RNase P.

### DETAILED DESCRIPTION

Provided in the present application, in certain aspects, is a nucleic acid purification method comprising a wash step comprising use of a water-immiscible solvent. The present application is based, at least in part, on the inventor's findings demonstrating that use of a water-immiscible wash step eliminates the need for numerous and lengthy wash steps found in conventional nucleic acid purification techniques. In some instances, only a single wash step comprising use of a water-immiscible solvent is needed in the nucleic acid purification method. The nucleic acid purification methods described herein can be completed in a rapid manner as only a single wash step is needed, and the drying step often performed prior an elution step in a conventional nucleic acid purification method is not needed. Additionally, the use of a water-immiscible solvent as a wash allows for the integration of a wash step with another step further enhancing the speed and efficiency of the methods (e.g., such as by forming a two-phase solution comprising a layer of the water-immiscible solvent and a layer comprising another fluid, such as for binding the nucleic acids to a nucleic acid binding medium or another wash solution, that are passed through the nucleic acid binding medium in a single centrifugation step). In addition to the time efficiency of the methods provided herein, the use of a water-immiscible wash step improves the removal of inhibitors of downstream processes, such as PCR, as compared to conventional washes not using a water-immiscible solvent. The resulting elution step eluate obtained from a nucleic acid purification method described herein contains few inhibitors and thus a greater amount of the elution step eluate may be used, without dilution, in a downstream process such as PCR, thereby further improving the speed and sensitivity of such a process.

Thus, in some aspects, provided herein is a method of purifying a nucleic acid from a sample, the method comprising a wash step comprising use of a water-immiscible solvent (e.g., dimethyl octanol), such as applied to a nucleic acid bound to a nucleic acid binding medium.

In other aspects, provided herein is a method of purifying a nucleic acid from a sample, the method comprising: (a) forming a two-phase solution on top of a silica medium, wherein the two-phase solution comprises a first layer comprising an aqueous binding solution and the sample containing the nucleic acid, wherein the two-phase solution comprises a second layer comprising a water-immiscible solvent, and wherein the first layer is in contact with the silica medium and the second layer is on top of the first layer; (b) passing the two-phase solution through the silica medium; (c) loading an elution solution on top of the silica medium; and (d) passing the elution solution through the silica medium to obtain the purified nucleic acid.

In other aspects, provided herein is a composition comprising a nucleic acid, wherein the composition is obtained from a nucleic acid purification method described herein. In some embodiments, the composition obtained from the nucleic acid purification method described herein has a lower amount, such as at least about 2 fold less, of one or more compounds inhibiting nucleic acid amplification (e.g., PCR inhibitors) as compared to a composition obtained from a nucleic acid purification method not comprising use of a wash step comprising a water-immiscible solvent.

### A. Definitions

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### B. Nucleic Acid Purification Methods

In certain aspects, provided herein is a method of purifying a nucleic acid from a sample, the method comprising a wash step comprising use of a water-immiscible solvent.

The methods described herein may be completed in various ways and formats. For example, in some embodiments, the method accomplishes certain tasks, such as a nucleic acid binding task wherein a nucleic acid from a sample is bound by a nucleic acid binding medium, a wash task wherein the nucleic acid binding medium is washed to remove components of the sample, such as inhibitors of downstream processes, and an elution task wherein the nucleic acid is eluted from the nucleic acid binding medium. In some embodiments, each task of the method is completed in an independent step. In some embodiments, more than one of the tasks (or at least a portion thereof) are integrated. For example, in some embodiments, the wash task is integrated, at least in part, with another step of the purification method, such as the nucleic acid binding task, e.g., via the formation of a two-phase solution and passing the two-phase solution through a nucleic acid binding membrane or filter in a single centrifugation step. Description of certain tasks and steps in a modular fashion is not intended to limit the scope of the disclosure, and one of ordinary skill in the art will readily appreciate that certain tasks and steps can be integrated, at least in part, based on the teachings provided herein. Unless otherwise stated, reference to a specific step, such as a nucleic acid binding step or a wash step, is not intended to mean that the step must be completed without integration of other tasks and/or steps described herein.

The methods described herein are amendable to being performed in both low-throughput and high-throughput formats. For example, in some embodiments, the nucleic acid purification method is applied to a plurality of samples and/or replicates in concert, e.g., using a multi-welled plate such as a 96-well plate or a 384-well plate. In some embodiments, the method is performed in a column format (e.g., a column comprising a packed nucleic acid binding medium, or a nucleic acid binding membrane or filter) such that tasks and/or steps are completed by loading a composition, such as a fluid, on top of the nucleic acid binding medium and then passing the composition through the nucleic acid binding medium by application of a force. For column formats, the passing of a composition through the nucleic acid binding medium can be completed in a variety of manners. In some embodiments, the composition is passed through the nucleic acid binding medium using a centrifugation technique. In some embodiments, the composition is passed through the nucleic acid binding medium using a pressure-based technique, such as a positive pressure technique or a negative pressure technique.

In some embodiments, the nucleic acid binding medium, such as a silica medium, is positioned in a container (such as a well of a multi-welled plate) and configured with space for loading a composition, such as a fluid, on top of the nucleic acid binding medium and with space for capturing an eluate below the nucleic acid binding medium.

A variety of centrifugation speeds and times may be utilized to pass a composition, such as a fluid, through a nucleic acid binding medium, such as a silica membrane or filter. One of ordinary skill in the art will appreciate that centrifugation speeds and time may be optimized for efficiency (including time efficiency and purification efficiency) and to ensure that all fluid has passed through the nucleic acid binding medium. In some embodiments, the centrifugation is performed at about 1,000×g to about 5,000×g, such as any of about 2,000×g to about 4,000×g, or about 2,500×g to about 3,500×g. In some embodiments, the centrifugation is performed for about 1 minute to about 10 minutes, such as about any of about 3 minutes to about 7 minutes, about 2 minutes to about 4 minutes, or about 6 minutes to about 8 minutes. In some embodiments, the centrifugation is performed for less than about 10 minutes, such as less than about any of 9.5 minutes, 8.5 minutes, 8 minutes, 7.5 minutes, 7 minutes, 6.5 minutes, 6 minutes, 5.5 minutes, 5 minutes, 4.5 minutes, 4 minutes, 3.5 minutes, 3 minutes, 2.5 minutes, 2 minutes, 1.5 minutes, 1 minute, or 0.5 minutes. In some embodiments, the centrifugation is performed for about any of 10 minutes, 9.5 minutes, 8.5 minutes, 8 minutes, 7.5 minutes, 7 minutes, 6.5 minutes, 6 minutes, 5.5 minutes, 5 minutes, 4.5 minutes, 4 minutes, 3.5 minutes, 3 minutes, 2.5 minutes, 2 minutes, 1.5 minutes, 1 minute, or 0.5 minutes.

In some embodiments, the method is performed in a magnetic particle-based format (e.g., using silica magnetic particles). In such embodiments, a magnetic force may be applied to a container comprising the magnetic particles to pellet the magnetic particles thus allowing for removal of a surrounding composition, such as a fluid (i.e., a supernatant).

In some embodiments, the method is performed using a device, such as a microfluidic device, configured to perform the method described herein.

In some embodiments, the method is performed at a temperature of about 20° C. to about 37° C., such as any of 20° C., 21° C., 22° C., 23° C., 24° C., 25° C., 26° C., 27° C., 28° C., 29° C., 30° C., 31° C., 32° C., 33° C., 34° C., 35° C., 36° C., or 37° C. In some embodiments, the method is performed at an ambient temperature, such as room temperature.

In some embodiments, the nucleic acid purification method (from sample to obtaining the purified nucleic acid) is completed in about 5 minutes to about 40 minutes, such as any of about 10 minutes to about 30 minutes, about 15 minutes to about 25 minutes, or about 15 to about 20 minutes. In some embodiments, the nucleic acid purification method (from sample to obtaining the purified nucleic acid) is completed in less than about 40 minutes, such as less than about any of 35 minutes, 34 minutes, 33 minutes, 32 minutes, 31 minutes, 30 minutes, 29 minutes, 28 minutes, 27 minutes, 26 minutes, 25 minutes, 24 minutes, 23 minutes, 22 minutes, 21 minutes, 20 minutes, 19 minutes, 18 minutes, 17 minutes, 16 minutes, 15 minutes, 14 minutes, 13 minutes, 12 minutes, 11 minutes, or 10 minutes.

In some embodiments, the method described herein are useful for obtaining a purified nucleic acid. In some embodiments, reference to a purified nucleic acid is intended to reflect that the nucleic acids containing a composition, such as an eluate, have been processed by a nucleic acid purification method described herein. In some embodiments, the composition comprising a purified nucleic acid will contain other substances, such as an inhibitor.

i. Samples

The methods disclosed herein are useful for a diverse array of samples containing, or suspected of containing, a nucleic acid.

In some embodiments, the sample comprises a bodily fluid, such as a sample comprising a blood sample, serum sample, convalescent plasma sample, oropharyngeal sample, including that obtained from an oropharyngeal swab, nasopharyngeal sample, including that obtained from a nasopharyngeal swab, buccal sample, bronchoalveolar lavage sample, including that obtained from an endotracheal aspirator, a urine sample, a sweat sample, a sputum sample, a salivary sample, a tear sample, a bodily excretion sample, or cerebrospinal fluid sample. In some embodiments, the sample comprise a solid, such as a sample comprising a fecal sample.

In some embodiments, the sample is from an individual. In some embodiments, the individual is a mammal, such as a human, bovine, horse, feline, canine, rodent, or primate. In some embodiments, the method comprises obtaining the sample from an individual.

In some embodiments, the sample comprises an environment sample, such as a sample comprising remains of in individual, such as a human or an animal, a food, a microorganism, a plant or its components, soil, sediment, rock, reef, sludge, decomposing biological matter, archaeological remains, oil, water, or air or particulates therein.

In some embodiments, the sample is suspected of containing, and in some instances contains, a nucleic acid associated with or originating from a target pathogen. In some embodiments, the target pathogen is a pathogen associated with a disease of an individual, such as a human disease. In some embodiments, the target pathogen is a bacterium, such as E. coli, Streptococcus, or Salmonella. In some embodiments, the target pathogen is a virus. In some embodiments, the virus is of the Coronaviridae family. In some embodiments, the virus is of the Betacoronavirus genus. In some embodiments, the virus is of the Sarbecovirus subgenus. In some embodiments, the virus is of the SARSr-CoV species. In some embodiments, the virus is a SARS-CoV strain. In some embodiments, the virus is selected from the group consisting of Severe Acute Respiratory Syndrome coronavirus 2 (SARS-CoV-2), Severe Acute Respiratory Syndrome coronavirus (SARS-CoV), Bat SARS-like coronavirus WIV1 (Bat SL-CoV-WIV1), alpha coronaviruses 229E (HCoV-229E), New Haven coronavirus NL63 (HCoV-NL63), beta coronaviruses 0C43 (HCoV-0C43), coronavirus HKU1 (HCoV-HKU1), and Middle East Respiratory Syndrome coronavirus (MERS-CoV).

In some embodiments, the nucleic acid comprises a RNA or DNA, or any combination thereof. In some embodiments the nucleic acid is a RNA or DNA. In some embodiments, the RNA is mRNA, tRNA, rRNA, or iRNA. In some embodiments, the RNA is a double-stranded viral RNA. In some embodiments, the RNA is a single-stranded viral RNA. In some embodiments, the RNA is negative-sense RNA, positive-sense RNA, or ambisense RNA.

In some embodiments, the sample is processed prior to subjecting to a nucleic acid purification method described herein. For example, in some embodiments, the sample is subjected to any one or more of a flocculation technique, a protein precipitation technique, a pathogen inactivation technique.

ii. Nucleic Acid Binding Steps

The methods of purifying a nucleic acid described herein comprises a nucleic acid binding step wherein the nucleic acids in a sample are bound by a nucleic acid binding medium. In some embodiments, the nucleic acid binding medium is in the form of a filter and/or fiber, such as a membrane. In some embodiments, the nucleic acid binding medium is in the form of a particle, such as a packed particulate column or magnetic particles.

In some embodiments, the nucleic acid binding medium is a silica-based medium. In some embodiments, the nucleic acid binding medium is selected from the group consisting of a silica membrane or filter (such as a microfiber), silica slurry, silica particulate, silicate glass (such as a powder, microbead, flint glass, borosilicate glass, or a glass fiber), silica gel, silica magnetic bead, and diatomaceous earth.

In some embodiments, the method comprises use of a binding solution (e.g., an aqueous binding solution comprising an aqueous component). For example, in some embodiments, the method comprises mixing the sample (suspected of containing a nucleic acid, and in some instances containing a nucleic acid) and a binding solution. In some embodiments, the mixture of the sample and the binding solution are subjected to a nucleic acid binding medium. In some embodiments, the binding solution facilitates the binding of a nucleic acid (such as RNA and/or DNA) to the nucleic acid binding medium. In some embodiments, the binding solution facilitates (such as also facilitates) another action, such as cell lysis. In some embodiments, the sample is admixed with a first binding solution, and then admixed with a second binding solution. For example, in some embodiments, the sample is admixed with a first binding solution, such as a binding solution comprising a chaotropic agent, e.g., a guanidinium salt, and then the sample admixed with the first binding solution comprising a chaotropic agent is admixed with a second binding solution, such as a binding solution comprising a short-chain alcohol and, optionally, a reducing agent. Binding solution are well known in the art. See Vandeventer et al., J Phys Chem B, 117, 2013; and Honsvall & Robertson, J Biomol Tech, 28, 2017, which are hereby incorporated herein by reference in their entirety. In some embodiments, the binding solution comprises a chaotropic salt. In some embodiments, the chaotropic salt is a guanidinium salt. In some embodiments, the guanidinium salt is guanidinium thiocyanate, guanidinium chloride (guanidinium HCl), or a combination thereof. In some embodiments, the chaotropic salt is selected from the group consisting of a lithium salt (such as lithium perchlorate, lithium acetate, or a combination thereof), a magnesium salt (such as magnesium chloride), a sodium salt (such as sodium dodecyl sulfate or sodium iodide), a potassium salt (such as potassium acetate), urea, and thiourea. In some embodiments, the binding solution comprises the chaotropic salt at a concentration of about 8 M to about 1 M, such as any of about 8 M to about 5 M, about 7 M to about 5 M, about 6.5 M to about 5 M, or about 3.5 M to about 4.5 M. In some embodiments, the binding solution comprises the chaotropic salt at a concentration of about 8 M or less, such as about any of 7.5 M or less, 7 M or less, 6.5 M or less, 6 M or less, 5.5 M or less, 5 M or less, 4.5 M or less, 4 M or less, 3.5 M or less, 3 M or less, 2.5 M or less, 2 M or less, 1.5 M or less, or 1 M or less. In some embodiments, the binding solution comprises the chaotropic salt at a concentration of about 1 M or more, such as about 1.5 M or more, 2 M or more, 2.5 M or more, 3 M or more, 3.5 M or more, 4 M or more, 4.5 M or more, 5 M or more, 5.5 M or more, 6 M or more, 6.5 M or more, 7 M or more, 7.5 M or more, or 8 M or more. In some embodiments, the binding solution comprises the chaotropic salt at a concentration of about any of 1 M or more, 1.5 M, 2 M, 2.5 M, 3 M, 3.5 M, 4 M, 4.5 M, 5 M, 5.5 M, 6 M, 6.5 M, 7 M, 7.5 M, or 8 M.

In some embodiments, the binding solution (such as the aqueous binding solution) comprises a short-chain alcohol. In some embodiments, the short-chain alcohol is ethanol, methanol, propanol, isopropanol (e.g., 2-propanol), or a combination thereof. In some embodiments, the short-chain alcohol is ethanol. In some embodiments, the short-chain alcohol has a concentration of about 1% to about 100%. In some embodiments, the aqueous binding solution comprises the short-chain alcohol at a concentration of about 5% to about 80%, such as about 30% to about 75%.

In some embodiments, the binding solution comprises a chaotropic salt and a short-chain alcohol. In some embodiments, the ratio of the chaotropic salt and the short-chain alcohol is about 3 to about 0-4 (based on volume), such as any of about 3 to about 1, about 3 to about 2, about 3 to about 3, and about 3 to about 4.

In some embodiments, the binding solution comprises one or more other agents. In some embodiments, the other agent of the binding solution is a reducing agent. In some embodiments, the reducing agent is selected from the group consisting of dithiothreitol (DTT), tris(2-carboxyethyl)phosphine (TCEP), sodium sulfate, dithioerythritol (DTE), 2-mercaptoethanol ((3-mercaptoethanol; BME). In some embodiments, the other agent of the binding solution is a buffer, such as a Tris, e.g., Tris-HCl. In some embodiments, the binding solution has a pH of about 4 to about 8, such as any of about 5 to about 8, about 5.5 to about 7.5, or about 6 to about 7.

In some embodiments, the binding solution comprises a short-chain alcohol, such as ethanol, and a reducing agent, such as TCEP.

In some embodiments, the nucleic acid binding step comprises loading a binding solution admixing with a sample on or with a nucleic acid binding medium. In some embodiments, the nucleic acid binding step comprises passing the binding solution admixed with the sample through the nucleic acid binding medium. In some embodiments, the nucleic acid binding step comprises admixing the binding solution, the sample, and the nucleic acid binding medium. In some embodiments, the nuclei acid binding step comprises separating (such as by removing) the binding solution admixed with the sample and the nucleic acid binding medium, such as after mixing and/or incubation.

iii. Washing Steps

The methods of purifying a nucleic acid described herein comprise use of a water-immiscible solvent in a wash step. In some embodiments, the wash task completed during the nucleic acid purification method described herein consists essentially of a water-immiscible solvent wash step. In some embodiments, the wash step is an independently performed step (e.g., a water-immiscible solvent is loaded on top of a silica medium and passed through the silica medium without integration of another step described herein). In some embodiments, the wash step is completed in conjunction with another step, such as a nucleic acid binding step (e.g., use of a two-phase solution comprising a layer comprising a binding solution admixed with a sample and layer comprising a water-immiscible solvent, which are passed through a silica membrane in a single processing step, such as a single centrifugation step).

In some embodiments, the water-immiscible solvent comprises a long-chain aliphatic alcohol. In some embodiments, the water-immiscible solvent comprises an octanol, nonanol, decanol, nerol, undecanol, or a mixture thereof. In some embodiments, the octanol is dimethyl-octanol, which is also known as 3,7-dimethyl-1-octanol, 3,7-dimethyloctan-1-ol, tetrahydrogeraniol, pelargol, dihydrocitronellol, perhydrogeraniol, and geraniol tetrahydride. In some embodiments, the octanol is selected from the group consisting of 1-octanol, 2-octanol, and 2-ethylhexanol. In some embodiments, the nonanol is selected from the group consisting of 1-nonanol and 2-nonanol. In some embodiments, the decanol is selected from the group consisting of 1-decanol (also known as decyl alchol) and 5-decanol. In some embodiments, the undecanol is selected from the group consisting of 1-undecanol (undecan-1-ol), 2-undecanol, and 5-undecanol.

In some embodiments, the water-immiscible solvent used in a wash step, e.g., such as applied to a nucleic acid binding medium, has a concentration of about 80% to about 100%, such as about 80% to about 90%, about 85% to about 95%, or about 90% to about 100%. For example, in some embodiments, the wash applied to a nucleic acid binding medium comprises a water-immiscible solvent (such as dimethyl-octanol) at a concentration of about 80% to about 100%, wherein the water-immiscible solvent is comprises a dilutant, such as water, an organic solvent, such as an alcohol, or a buffer. In some embodiments, the concentration of the water-immiscible solvent is least about 80%, such as at least about any of 85%, 90%, 95%, or 100%. In some embodiments, the concentration of water-immiscible solvent is less than about 100%, such as less than about any of 95%, 90%, 85%, or 80%. In some embodiments, the concentration of the water-immiscible solvent is about any of 80%, 85%, 90%, 95%, or 100%.

In some embodiments, the water-immiscible solvent has a water solubility of about 10 g or less, such as about any of 5 g or less, 4 g or less, 3 g or less, 2 g or less, 1 g or less, 0.5 g or less, 0.1 g or less, 0.05 g or less, or 0.001 g or less, per 100 mL of water.

In some embodiments, the wash step comprises loading a water-immiscible solvent on or with a nucleic acid binding medium. In some embodiments, the wash step comprises passing the water-immiscible solvent through the nucleic acid binding medium. In some embodiments, the wash step comprises admixing the water-immiscible solvent and the nucleic acid binding medium. In some embodiments, the wash step comprises separating (such as by removing) the water-immiscible solvent and the nucleic acid binding medium, such as after mixing and/or incubation.

In some embodiments, the method further comprises a wash step using a wash not comprising a water-immiscible solvent. In some embodiments, the wash comprises the binding solution or a dilute form thereof. In some embodiments, the wash comprises a buffered solution, such as a Tris buffered solution.

In some embodiments, the method does not comprise a wash step using a wash not comprising a water-immiscible solvent. In some embodiments, the wash steps of the method consist essentially of water-immiscible solvent wash steps. In some embodiments, the wash task is completed using more than one wash step, wherein at least one wash step is a water-immiscible solvent wash step. In some embodiments, when more than one wash step is used in the method, two or more wash steps may be integrated, at least in part (e.g., a water-immiscible solvent and another water fluid are formed in layers and passed through a nucleic acid binding medium in a single process).

In some embodiments, the water-immiscible solvent exhibits some degree of an inhibitory effect on a downstream process, such as acting as a PCR inhibitor. Such water-immiscible solvents may still be used in the methods described herein. For example, in some embodiments, the water-immiscible solvent is compatible with a downstream process using the purified nucleic acids obtained from the methods described herein. In some embodiments, the water-immiscible solvent wash step is followed by another wash step comprising use of a non-water immiscible wash step and/or a water-immiscible solvent compatible with a downstream process.

In some embodiments, the water-immiscible solvent comprises a water-immiscible ester (e.g., a fatty-acid ester or any ester with a plurality of carbon atoms, such as butyl hexanote or hexyl hexanoate), geraniol, a water-immiscible oil, mineral oil, paraffin oil, fluorocarbon oil, perfluorocarbon fluids, perfluorodecalin, perfluoroperhydrophenanthrene, perfluorooctylbromide, palm oil, coconut oil, canola oil, soybean oil, sunflower oil, rapeseed oil, peanut oil, cotton seed oil, palm kernel oil, olive oil, a water-immiscible silicone oil, such as dimethyl silicone fluid or fluorosilicone, caprylic acid, capric acid, lauric acid, myristic acid, palmitic acid, stearic acid, cis-oleic acid, trans-oleic acid, myristoleic acid, palmitoleic acid, sapienic acid, oleic acids, elaidic acid, vaccenic acid, linoleic acid, linoelaidic acid, α-linolenic acid, or ricinoleic acid, or a combination thereof.

iv. Two-Phase Solutions Comprising a First Layer and a Second Layer

In some embodiments, two or more tasks and/or steps are integrated, at least in part, by forming a two-phase solution comprising a first layer and a second layer. In such embodiments, one or more characteristics of a layer, such as solubility, miscibility, or density, are used to maintain a separation of the layers, at least to a degree, such that a nucleic acid binding medium can be exposed to layers in series (such as when subjecting the system to centrifugation). Such layered systems are not limited to having only two layers and/or two phases. In some embodiments, the interface between two layers may comprise some mixing of the two layers.

In some embodiments, forming the two-phase solution comprises loading a first layer on top of the nucleic acid binding medium, such as a silica medium, and then loading a second layer on top of the first layer. In some embodiments, the first layer is a binding solution (such as an aqueous binding solution) admixed with a sample. In some embodiments, the method further comprises obtaining a first layer by admixing a binding solution and a sample. In some embodiments, the two-phase solution is passed through a nucleic acid binding medium by subjecting the system to centrifugation or the application of a pressure, such as positive pressure or negative pressure. In some embodiments, passing the two-phase solution through the nucleic acid binding medium, such as a silica medium, is completed in a single centrifugation step.

v. Drying Step

Generally, drying steps may be used in a nucleic acid purification technique to remove any unwanted solution from the system prior to an elution step. For instance, a drying step may be used to reduce the carryover of a certain solvent and/or compounds inhibiting nucleic acid amplification (e.g., PCR inhibitors) to the purified product comprising a nucleic acid. In some embodiments, when a filter or column-based technique is utilized, the drying step comprises a centrifugation or a pressure-based step wherein no additional solution or solvent is added to the filter or column prior to complete the drying process, such as completed via a centrifugation or the application of pressure.

In some embodiments, the method does not comprise an independent drying step. In some embodiments, the method does not comprise a drying step between a wash step comprising use of a water-immiscible solvent and an elution step.

vi. Elution steps

The methods of purifying a nucleic acid described herein comprise use of an elution solution in an elution step.

In some embodiments, the elution solution comprises a hyposmotic solution. In some embodiments, the elution solution is a hyposmotic solution. In some embodiments, the elution solution comprises deionized water. In some embodiments, the elution solution is deionized water. In some embodiments, the elution solution comprises ultrapure water. In some embodiments, the elution solution is ultrapure water. In some embodiments, the elution solution comprises DNase and RNase-free water. In some embodiments, the elution solution is DNase and RNase-free water. In some embodiments, the elution solution comprises a low ionic strength buffered solution. In some embodiments, the elution solution is a weak buffer, such as a 100 mM or less Tris or Tris and EDTA buffer.

In some embodiments, the elution step comprises loading an elution solution on or with a nucleic acid binding medium. In some embodiments, the elution step comprises passing the elution solution through the nucleic acid binding medium. In some embodiments, the elution step comprises admixing the elution solution and the nucleic acid binding medium. In some embodiments, the elution step comprises separating (such as by removing) the elution solution and the nucleic acid binding medium, such as after mixing and/or incubation.

In some embodiments, the method comprises collecting an eluate comprising the elution solution in a clean container, such as a container not contaminated by previous steps.

vii. Additional Method Steps

In certain aspects, the nucleic acid purification methods described herein are integrated with one or more upstream and/or downstream methods.

In some embodiments, the method comprises obtaining a sample. In some embodiments, the sample is a bodily fluid sample, such as a blood sample, serum sample, convalescent sample, oropharyngeal sample, including that obtained from an oropharyngeal swab, nasopharyngeal sample, including that obtained from a nasopharyngeal swab, buccal sample, or bronchoalveolar lavage sample, including that obtained from an endotracheal aspirator. In some embodiments, the obtained sample is processed prior to subjecting to a nucleic acid purification method described herein, such as to obtain at least a portion of the sample from a collection device, aliquot portions of the sample, and/or inactivate any pathogenic components in the sample.

In some embodiments, the eluate obtained from a nucleic acid purification method described herein is processed to identify and, if desired, quantify nucleic acids in the eluate. In some embodiments, the method comprises a PCR technique, such as real-time PCR (rt-PCR), quantitative PCR (qPCR), reverse transcription PCR (RT-PCR), or digital PCR (such as droplet digital PCR). In some embodiments, the method comprises sequencing the nucleic acids in the eluate, such as using a next-generation sequencing technique.

In some embodiments, the eluate obtained from a nucleic acid purification method described herein can be used to reconstitute a lyophilized pellet comprising reagents useful for amplification, for example PCR reagents. In some embodiments, such technique allows for the maximization of sample input into a reaction, such as a PCR reaction.

In some embodiments, the method comprises assessing whether the sample contains the presence (or lacks the presence) of a certain nucleic acid. In some embodiments, the nucleic acid is from a pathogen. In some embodiments, the pathogen is a virus, e.g., SARS-CoV-2. In some embodiments, the pathogen is a bacterium.

viii. Exemplary Nucleic Acid Purification Methods

Exemplary nucleic acid purification methods are described below using steps described herein. Description of specific nucleic acid purification methods in this section is not to be construed as limiting the nucleic acid purification methods encompassed by the disclosure of the instant application.

In some aspects, provided is a method of purifying a nucleic acid from a sample, the method comprising: (a) forming a two-phase solution on top of a nucleic acid binding medium, such as a silica medium, wherein the two-phase solution comprises a first layer comprising an aqueous binding solution and the sample containing the nucleic acid (or suspected of containing a nucleic acid), wherein the two-phase solution comprises a second layer comprising a water-immiscible solvent, and wherein the first layer is on top of the nucleic acid binding medium, such as in contact with the nucleic acid binding medium, and the second layer is on top of the first layer; (b) passing the two-phase solution through the nucleic acid binding medium; (c) loading an elution solution on top of the nucleic acid binding medium; and (d) passing the elution solution through the nucleic acid binding medium to obtain the purified nucleic acid. In some embodiments, the nucleic acid binding medium, such as a silica medium, is a silica filter or membrane. In some embodiments, the nucleic acid binding medium is in a container (such as a well of a multi-welled plate) and configured with space for loading a fluid on top of the nucleic acid binding medium and with space for capturing a fluid eluate below the nucleic acid binding medium. In some embodiments, passing the two-phase solution and passing the elution solution through the nucleic acid binding medium comprises subjecting the nucleic acid binding medium and any fluid, if present, such as a two-phase solution or an elution solution, to a centrifugation or pressure-based technique.

In some embodiments, the method comprises: (a) admixing a sample suspected of containing a nucleic acid and a binding solution comprising a chaotropic salt and, optionally, a short-chain alcohol (e.g., ethanol) and/or reducing agent (e.g., DTT or TCEP); (b) loading the admixed sample and binding solution on a silica membrane or filter to form a first layer of a two-phase solution; (c) loading a water-immiscible solvent (e.g., dimethyl-octanol) on top of the first layer to form the two-phase solution; (d) passing the two-phase solution through the silica membrane or filter, such as using a centrifugation or pressure-based technique; (e) loading an elution solution (e.g., deionized water) on top of the silica membrane or filter; and (f) passing the elution solution through the silica membrane or filter, such as using a centrifugation or pressure-based technique, to obtain the purified nucleic acid. In some embodiments, the method comprises collecting the eluate obtained from passing the elution solution through the silica membrane or filter. In some embodiments, the silica membrane or filter is in a container (such as a well of a multi-welled plate) and configured with space for loading a fluid on top of the silica membrane or filter and with space for capturing a fluid eluate below the silica membrane or filter.

In some aspects, provided is a method of purifying a nucleic acid from a sample, the method comprising: (a) loading a binding solution (such as an aqueous binding solution) admixed with the sample containing a nucleic acid (or suspected of containing a nucleic acid) on top of a nucleic acid binding medium, such as a silica medium; (b) passing the binding solution admixed with the sample through the nucleic acid binding medium; (c) loading a water-immiscible solvent on top of the nucleic acid binding medium; (d) passing the water-immiscible solvent through the nucleic acid binding medium; (e) loading an elution solution on top of the nucleic acid binding medium; and (f) passing the elution solution through the nucleic acid binding medium to obtain the purified nucleic acid. In some embodiments, the nucleic acid binding medium (e.g., a silica medium) is a silica filter or membrane. In some embodiments, the nucleic acid binding medium is in a container (such as a well of a multi-welled plate) and configured with space for loading a fluid on top of the silica medium and with space for capturing a fluid eluate below the nucleic acid binding medium. In some embodiments, passing the binding solution admixed with the sample, passing the water-immiscible solvent, and passing the elution solution through the nucleic acid binding medium comprises subjecting the nucleic acid binding medium and any fluid, if present, such as the binding solution admixed with the sample, the water-immiscible solvent, or the elution solution, to a centrifugation or pressure-based technique.

In some embodiments, the method comprises: (a) admixing a sample suspected of containing a nucleic acid and a binding solution (such as an aqueous binding solution) comprising a chaotropic salt and, optionally, a short-chain alcohol (e.g., ethanol) and/or reducing agent (e.g., DTT or TCEP); (b) loading the binding solution admixed with the sample on a silica membrane or filter, such as using a centrifugation or pressure-based technique; (c) loading a water-immiscible solvent (e.g., dimethyl-octanol) on top of the silica membrane or filter; (d) passing the water-immiscible solvent through the silica membrane or filter, such as using a centrifugation or pressure-based technique; (e) loading an elution solution (e.g., deionized water) on top of the silica membrane or filter; and (f) passing the elution solution through the silica membrane or filter, such as using a centrifugation or pressure-based technique, to obtain the purified nucleic acid. In some embodiments, the method comprises collecting the eluate obtained from passing the elution solution through the silica membrane or filter. In some embodiments, the silica membrane or filter is in a container (such as a well of a multi-welled plate) and configured with space for loading a fluid on top of the silica membrane or filter and with space for capturing a fluid eluate below the silica membrane or filter.

In some aspects, provided is a method of purifying a nucleic acid from a sample, the method comprising: (a) loading a binding solution (such as an aqueous binding solution) admixed with the sample containing a nucleic acid (or suspected of containing a nucleic acid) on top of a nucleic acid binding medium, such as a silica medium; (b) passing the binding solution admixed with the sample through the nucleic acid binding medium; (c) loading a wash solution comprising a short-chain alcohol, e.g., ethanol, on top of the nucleic acid binding medium; (d) passing the wash solution through the nucleic acid binding medium; (e) loading a water-immiscible solvent on top of the nucleic acid binding medium; (f) passing the water-immiscible solvent through the nucleic acid binding medium; (g) loading an elution solution on top of the nucleic acid binding medium; and (h) passing the elution solution through the nucleic acid binding medium to obtain the purified nucleic acid. In some embodiments, the nucleic acid binding medium (e.g., a silica medium) is a silica filter or membrane. In some embodiments, the nucleic acid binding medium is in a container (such as a well of a multi-welled plate) and configured with space for loading a fluid on top of the silica medium and with space for capturing a fluid eluate below the nucleic acid binding medium. In some embodiments, passing the binding solution admixed with the sample, passing the wash solution, passing the water-immiscible solvent, and passing the elution solution through the nucleic acid binding medium comprises subjecting the nucleic acid binding medium and any fluid, if present, such as the binding solution admixed with the sample, the wash solution, the water-immiscible solvent, or the elution solution, to a centrifugation or pressure-based technique.

In some embodiments, the method comprises: (a) admixing a sample suspected of containing a nucleic acid and a binding solution (such as an aqueous binding solution) comprising a chaotropic salt and, optionally, a short-chain alcohol (e.g., ethanol) and/or reducing agent (e.g., DTT or TCEP); (b) loading the binding solution admixed with the sample on a silica membrane or filter, such as using a centrifugation or pressure-based technique; (c) loading a wash solution comprising a short-chain alcohol, e.g., ethanol, on top of the silica membrane or filter; (d) passing the wash solution through the silica membrane or filter, such as using a centrifugation or pressure-based technique; (e) loading a water-immiscible solvent (e.g., dimethyl-octanol) on top of the silica membrane or filter; (f) passing the water-immiscible solvent through the silica membrane or filter, such as using a centrifugation or pressure-based technique; (g) loading an elution solution (e.g., deionized water) on top of the silica membrane or filter; and (h) passing the elution solution through the silica membrane or filter, such as using a centrifugation or pressure-based technique, to obtain the purified nucleic acid. In some embodiments, the method comprises collecting the eluate obtained from passing the elution solution through the silica membrane or filter. In some embodiments, the silica membrane or filter is in a container (such as a well of a multi-welled plate) and configured with space for loading a fluid on top of the silica membrane or filter and with space for capturing a fluid eluate below the silica membrane or filter.

In some aspects, provided is a method of purifying a nucleic acid from a sample, the method comprising: (a) admixing a binding solution (such as an aqueous binding solution) with the sample containing a nucleic acid (or suspected of containing a nucleic acid) and a nucleic acid binding medium, such as a silica magnetic particle; (b) pelleting the nucleic acid binding medium and removing a resulting supernatant; (c) admixing the nucleic acid binding medium and a water-immiscible solvent; (d) pelleting the nucleic acid binding medium and removing a resulting supernatant; (e) admixing an elution solution and the nucleic acid binding medium; and (f) pelleting the nucleic acid binding medium to obtain the purified nucleic acid in a resulting supernatant. In some embodiments, pelleting the nucleic acid binding medium, such as a silica magnetic particle, comprises subject a container comprising the nucleic acid binding medium to a magnet for a period of time sufficient to pellet the nucleic acid binding solution.

In some aspects, provided is a method of purifying a nucleic acid from a sample, the method comprising: (a) admixing a binding solution (such as an aqueous binding solution) with the sample containing a nucleic acid (or suspected of containing a nucleic acid) and a nucleic acid binding medium, such as a silica magnetic particle; (b) pelleting the nucleic acid binding medium and removing a resulting supernatant; (c) admixing the nucleic acid binding medium and a wash solution comprising a short-chain alcohol, e.g., ethanol, and the nucleic acid binding medium; (d) pelleting the nucleic acid binding medium and removing a resulting supernatant; (e) admixing the nucleic acid binding medium and a water-immiscible solvent; (f) pelleting the nucleic acid binding medium and removing a resulting supernatant; (g) admixing an elution solution and the nucleic acid binding medium; and (h) pelleting the nucleic acid binding medium to obtain the purified nucleic acid in a resulting supernatant. In some embodiments, pelleting the nucleic acid binding medium, such as a silica magnetic particle, comprises subject a container comprising the nucleic acid binding medium to a magnet for a period of time sufficient to pellet the nucleic acid binding solution.

C. Compositions Obtained from the Purification Methods Described Herein

In other aspects, provided herein is a composition comprising a nucleic acid, wherein the composition is obtained from a nucleic acid purification method described herein. In some embodiments, the composition obtained from the nucleic acid purification method described herein has a lower amount, such as at least about 2 fold less, of one or more compounds inhibiting nucleic acid amplification (e.g., PCR inhibitors) as compared to a composition obtained from a nucleic acid purification method not comprising use of a wash step comprising a water-immiscible solvent. In some embodiments, the lower amount of one or more PCR inhibitors is reflected in the amount of elution eluate that may be used in a PCR reaction. In conventional purification techniques not comprising use of a wash step comprising a water-immiscible solvent, generally a maximum of about 5 μl of elution eluate template is added to a 20 μl PCR reaction as more than 5 μl leads to significant inhibition of PCR. In some embodiments, the composition (e.g., the elution solution eluate) obtained from a purification method described herein enables the use of about 5 μl to about 20 μl of the composition template to be used in a 20 μl PCR reaction as the presence of one or more PCR inhibitors is significantly reduced. In some embodiments, about 5 μl or more, such as about any of 6 μl or more, 7 μl or more, 8 μl or more, 9 μl or more, 10 μl or more, 11 μl or more, 12 μl or more, 13 μl or more, 14 μl or more, 15 μl or more, 16 μl or more, 17 μl or more, 18 μl or more, 19 μl or more, or 20 μl or more, of the elution solution template from a nucleic acid purification method described herein is used in a 20 μl PCR reaction. In some embodiments, the elution eluate template is not diluted prior to use in a PCR technique.

In some embodiments, the PCR inhibitor described herein is a component, such as a compound, that inhibits a features of a PCR reaction, such as an enzyme.

### D. Kits, Components, and Compositions (Such as Consumables)

In other aspects, the present disclosure provides kits, components, and compositions (such as consumables) useful for the methods described herein. For example, provided herein is a kit comprising a nucleic acid binding medium, such as a welled-plate comprising a silica membrane or filter, a wash solvent, such as a water-immiscible solvent, and an elution solution. In some embodiments, the kit comprises reagents for a downstream process using the purified nucleic acid, such as PCR reagents. In some embodiments, the components of the kits may come in separate containers. In some embodiments, the kit comprises instructions for use according the methods described herein.

### EXAMPLES

### Example 1

This example demonstrates the use of a nucleic acid purification method using a silica membrane, as described herein, to process a sample followed by reverse transcription PCR (RT-PCR) to amplify and detect SARS-CoV-2 RNA and human ribonuclease P (RNase P).

A 96-well filter plate comprising a silica membrane was obtained and attached to a 2-mL waste plate. Each 300 μl aliquot of a sample comprising guanidinium salt solution (4 M guanidinium SCN) suspected of containing a SARS-CoV-2 RNA was admixed with a binding solution comprising 300 μl of an ethanol-TCEP solution (1.167 mM TCEP in 100% ethanol). Each 600 μl admixed sample was added to an individual well of the 96-well plate. 250-300 μl of dimethyl octanol was pipetted on top of each 600 μl sample in a manner to create a two-phase solution comprising a first layer of the admixed sample on top of the silica membrane, and a second layer comprising dimethyl octanol on top of the first layer. The plate was sealed and then centrifuged at 3000×g for about 7 minutes.

The plate was removed from the centrifuge and the waste plate was replaced with a collection place. 70 μl of ultrapure water was added to each well on top of the silica membrane. The plate was sealed and then centrifuged at 3,000×g for about 3 minutes.

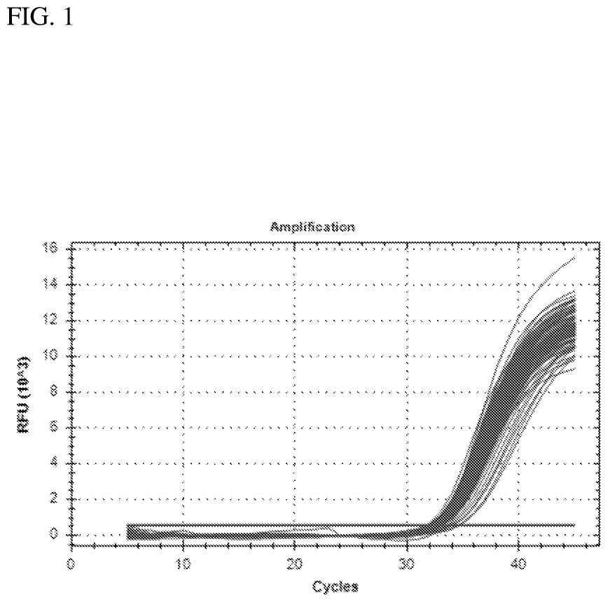

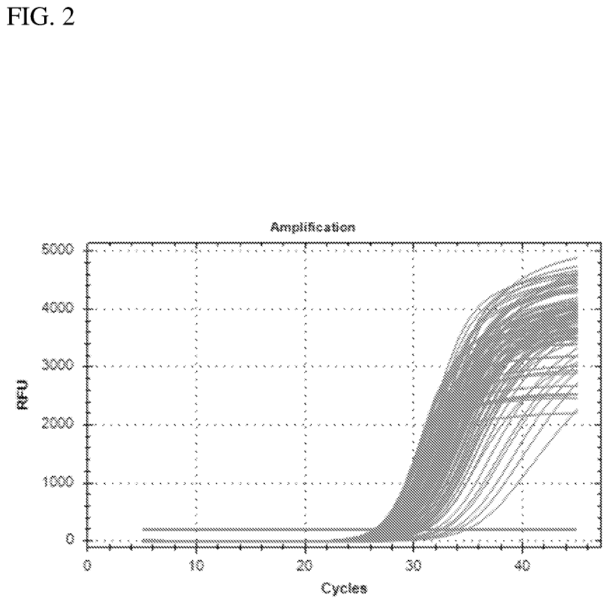

The plate was removed from the centrifuge and 15 μl of each eluate containing purified nucleic acids was added to 5.1 μl of a PCR mastermix for the detection of SARS-CoV-2 RNA and human RNase P. RT-PCR amplification and detection of amplified products was performed to detect the presence of SARS-CoV-2 RNA and human RNase P. RNase P serves an internal control to monitor sample quality, RNA extraction and purification, and for the detection of inhibitors of the PCR reaction. The relative fluorescence units (RFU) per cycle for the SARS CoV-2 viral RNA is shown in FIG. 1. The relative fluorescence units (RFU) per cycle for RNase P is shown in FIG. 2. As illustrated in FIG. 1 and FIG. 2, the amplified amount of human RNase P is significantly less than the amplified amount of SARS-CoV-2 RNA thus demonstrating that the nucleic acid purification method comprising a single wash step using a water-immiscible solvent results in a rapid and efficient purification method.

### Example 2

This example demonstrates the use of a nucleic acid purification method using magnetic particles, as described herein, to process a sample followed by reverse transcription PCR (RT-PCR) to amplify and detect SARS-CoV-2 RNA and human ribonuclease P (RNase P).

The sample lysates were prepared by adding aliquots of the samples to a 96-well plate and centrifuging at 3000× g for 1 minute. 150 μl of lysis buffer comprising guanidinium salt solution (4 M guanidinium SCN) was added to each well and mixed by pipetting or use of a shaker. The plate was then incubated for about 1 minute at room temperature. A 305 μl aliquot of Bind-Isopropanol (300 μl of isopropanol with 5 μl of Bind (BBD)) comprising magnetic particles was added to each well and mixed by pipetting or use of a shaker. The plate was then incubated for about 5 minutes at room temperature. The plate was then placed on a magnet for 10 minutes or until the supernatant in each well was clear. The supernatant was removed and discarded.

400 μl of freshly prepared 70% ethanol was added to each well without disturbing the pellet or mixing via pipetting. The plate was left on the magnet for 2 minutes or until the supernatant in each well was clear. The supernatant was removed and discarded.

300 μl of dimethyl octanol was added to each well without disturbing the pellet. The plate was left on the magnet for 2 minutes or until the supernatant in each well was clear. The supernatant was removed and discarded.

70 μl of ultrapure water was added to each well and the pellet was mixed by pipetting or use of a shaker. The plate was then incubated for about 5 minute at room temperature. The plate was then placed on a magnet for 2 minutes or until the supernatant in each well was clear. The supernatant was removed and retained for use in PCR reactions.

What is claimed is:

1. A method of purifying a nucleic acid from a sample, the method comprising:
(a) forming a two-phase solution on top of a silica medium,
wherein the two-phase solution comprises a first layer comprising an aqueous binding solution and the sample containing the nucleic acid,
wherein the two-phase solution comprises a second layer comprising a water-immiscible solvent, and
wherein the first layer is in contact with the silica medium and the second layer is on top of the first layer;
(b) passing the two-phase solution through the silica medium;
(c) loading an elution solution on top of the silica medium; and
(d) passing the elution solution through the silica medium to obtain the purified nucleic acid.

2. The method of claim 1, wherein forming the two-phase solution comprises loading the first layer on top of the silica medium and then loading the second layer on top of the first layer.

3. The method of claim 1 or 2, wherein the water-immiscible solvent comprises a long-chain aliphatic alcohol.

4. The method of any one of claims 1-3, wherein the water-immiscible solvent comprises an octanol, decanol, undecanol, or a mixture thereof.

5. The method of claim 4, wherein the water-immiscible solvent comprises dimethyl-octanol.

6. The method of any one of claims 1-5, further comprising obtaining the first layer by mixing the aqueous binding solution and the sample.

7. The method of any one of claims 1-6, wherein the aqueous binding solution comprises a chaotropic salt and/or a short-chain alcohol.

8. The method of claim 7, wherein the chaotropic salt is a guanidinium salt.

9. The method of claim 8, wherein the guanidinium salt is guanidinium thiocyanate or guanidinium chloride.

10. The method of any one of claims 7-9, wherein the short-chain alcohol comprises ethanol, methanol, propanol, isopropanol, or a combination thereof.

11. The method of claim 10, wherein the short-chain alcohol comprises ethanol.

12. The method of any one of claims 7-11, wherein the aqueous binding solution further comprises a reducing agent.

13. The method of claim 12, wherein the reducing agent is DTT or TCEP.

14. The method of any one of claims 1-13, wherein the silica medium is a silica membrane.

15. The method of any one of claims 1-13, wherein the silica medium is a silica-based filter.

16. The method of any one of claims 1-15, wherein the elution solution is deionized water.

17. The method of any one of claims 1-16, wherein the elution solution comprises a low ionic strength buffered solution.

18. The method of any one of claims 1-17, wherein the silica medium is positioned in a container, and wherein the container comprises a loading zone above the silica medium and an eluate zone below the silica medium.

19. The method of claim 18, wherein passing the two-phase solution through the silica medium or passing the elution solution through the silica medium comprises subjecting the container to centrifugation or the application of a pressure.

20. The method of claim 19, wherein passing the two-phase solution through the silica medium is completed in a single centrifugation step.

21. The method of any one of claims 1-20, wherein the method is completed in 20 minutes or less.

22. The method of any one of claims 1-21, wherein the method does not comprise a drying step between passing the two-phase solution through the silica medium and passing the elution solution through the silica medium.

23. A composition comprising a nucleic acid obtained from a nucleic acid purification method of any one of claims 1-22.

24. The composition of claim 23, wherein the composition comprises at least about 2 fold less of a compound inhibiting nucleic acid amplification as compared to a composition obtained from a nucleic acid purification method not comprising use of a wash step comprising a water-immiscible solvent.
